(19) United States
(12) Patent Application Publication
(10) Pub. No.: US 2003/0199922 A1
(43) Pub. Date: Oct. 23, 2003

(54) Pneumatic pressure bandage for medical applications

(76) Inventor: James S. Buckman, Vancouver, WA (US)

Correspondence Address: William S. Lovell, 6435 NE Neptune Dr., Lincoln City, OR, 97367, US

(21) Appl. No.: 10/128,600

(22) Filed: Apr. 22, 2002

(51) Int. Cl. A61B 17/00

(52) U.S. Cl. 606/202

(57) ABSTRACT

A pressure bandage applies a pressure pad to a dressing on a wound, or two or more such pressure pads are applied to the dressings of multiple wounds, and a pneumatically operated air bladder is placed over the pressure pad in each case and held in place by an over-wrapping bandage, the pressure bandage so installed then being used to apply amounts of pressure to respective one or more wounds as appropriate to the nature of each particular wound.

### CROSS REFERENCE TO RELATED APPLICATIONS

Not applicable

### STATEMENT REGARDING FEDERALLY SPONSORED RESEARCH OR DEVELOPMENT

Not applicable.

### BACKGROUND OF THE INVENTION

1. Field of the Invention

This invention relates to bandages, and specifically to pressure bandages that can be placed at essentially any location on a patient, human or animal, wherein dressings applied to wounds will require pressure to be provided thereto so as to bring about staunching of blood flow, or as a tourniquet, temporary holding of a hernia, and the like, and more particularly to pressure sources by which such bandages so placed over wounds, or in other circumstances requiring the application of pressure, can have pressure applied thereto.

2. Description of the Related Art

Quite a few patents have been issued relating to bandages, but Applicant has found none that encompass or suggest the subject matter of the present invention. The patents selected for study have included only those that, in one way or another, attempt to apply some kind of pressure or compression on a wound, or include other means for applying pressure to the body. In date order, latest first, these are as follows.

U.S. Pat. No. 6,315,745 issued Nov. 13, 2001, to Kloecker, describes a compressive garment intended to treat lymphedema and related illnesses that cause obstruction of lymphatic flow so as to cause an accumulation of lymph, particularly in the subcutaneous region so as to develop surface “ballooning,” at a wide variety of places on the body. The Kloecker device is applied over those areas at which such accumulations develop, which are most often in the limbs (but also around the pelvis), for the purpose of reducing the amount of accumulated fluid, and consists essentially of a compressive garment to be applied to the body extremities, the garment having a multiplicity of interconnected, cushion-like panels, each instance of which is custom made to fit onto a particular part of a particular limb and even of a particular patient, and includes interconnection means along the edges thereof for installation onto the patient. The device is applied to the limb, then held in place by those interconnection means, and compression air pressure is acquired by pumping air into all of the separate panels, either individually or having a single air inlet that connects within the device to all of the panels. The structure indicated preclude the use of a single instance of the device to a variety of places on the body, which is one feature of the present invention.

U.S. Pat. No. 6,316,686 issued Nov. 13, 2001, to Byrd, describes a medical pressure dressing comprising a frame including an aperture (“channel”) therein that is to be placed onto the patient's skin such that a wound or the like, and some amount of surrounding skin, lies below that aperture. Further included is a plunger disposed above that channel that can be forced downward thereon and held in such a pressure-applying disposition by overlaying the entire assembly with wraparound bandage.

U.S. Pat. No. 6,309,369 issued Oct. 30, 2001, to Lebovic, describes a binder for securing a bandage or dressing onto a relevant area of the torso, and is adapted to apply pressure on such bandage or dressing by securing onto the patient with wrap-around, elastic, and non-adhesive bandage.

U.S. Pat. No. 6,296,618 issued Oct. 2, 2001, to Gaber, describes a vest-like surgical compression garment that applies pressure to an underlying wound, or more commonly an operation site, by being secured to the body by way of flexible (i.e., “stretching,” fasteners between facing edges of the garment.

U.S. Pat. No. 6,235,043 issued May 22, 2001 to Reiley et al., describes an inflatable device that is inserted internally to a bone so as to force the marrow or medullary bone therein against the inner cortex of bones, and consists essentially of an inflatable but not expandable balloon of a pre-determined size and shape so to serve with particular bones.

U.S. Pat. No. 6,196,231 issued Mar. 6, 2001 to Reid, describes a device for treating lymphedema by applying pressure to those areas of the body, usually in the limbs, at which fluid accumulation has occurred. The device is specifically adapted to such treatment, and comprises an array of straps extending from a sleeve that is placed around the limb, one strap is loosened for insertion of an air bladder; air is pumped into the air bladder to achieve a desired pressure on the limb; the degree of tightening is determined and recorded using an indicia marker, the bladder is removed, the strap is re-tightened to effect the same degree of pressure on the limb, and the process is then repeated with respect to the other straps. The principal function of the device is first to determine the degree of hardness of the patient's limb, in terms of establishing the amount of pressure required to cause movement of the edema fluid, and once that information has been learned, the device, strap by strap, can be tightened to the degree of pressure required by that pre-determination, with the air bladder being removed as to each strap, and thereafter serves no purpose in applying continuing pressure to the limb.

U.S. Pat. No. 6,171,271 issued to Hörnberg on Jan. 9, 2001, describes a pressure bandage adapted specifically to hip joint prosthesis, and comprises an inelastic girdle shaped to enclose the pelvis area, and particularly the hip joint upon which operation has been carried out, and further an air bladder connected to a hand air pump and a pressure gauge whereby a desired degree of pressure can be applied to the hip.

U.S. Pat. No. 5,891,074 issued to Cesarczyk on Apr. 6, 1999, describes a pressure wound dressing including a pressure exerting support member and a sterile, pliant absorbent material that is placed against a wound, thereby achieving a pressure dressing against the wound. The degree of pressure being applied is determined from prior calibrations of the thickness exhibited by the pressure exerting support member when under particular pressure.

U.S. Pat. No. 6,799,650 issued to Harris on Sep. 1, 1998, describes a femoral compression device consisting essentially of a rigid restraining member that is strapped onto a patient to prevent hip flexion, and a compression strap afixed to that restraining member that is in turn applied to a compression element applied over a post-catheterization wound. No pneumatic devices are involved, pressure instead being applied by strap tightening.

U.S. Pat. No. 5,741,295 issued to McEwen on Apr. 21, 1998, describes a tourniquet cuff system, including an inflatable bladder for encircling and overlapping on itself around a limb, and is not directed towards application of pressure to a dressing on a wound.

U.S. Pat. No. 5,724,714 issued to Love on Mar. 10, 1998, describes a composite socket member for use with a prosthetic appliance to be applied to the residual part of a limb on which amputation has been carried out, wherein the socket member includes an air bladder that is integral to the socket member and serves to retain a good fit between the limb stump and the prosthetic device. The Love device is not directed towards application of pressure to a dressing on a wound.

U.S. Pat. No. 5,584,853 issued to McEwen on Dec. 17, 1996, describes a tourniquet apparatus related to McEwen '295, and is likewise not directed towards application of pressure to a dressing on a wound.

U.S. Pat. No. 5,234,459 issued to Hans Lee on Aug. 10, 1993, describes a method for controlling the flow of blood through the forearm by use of a tourniquet, consisting essentially of a strap that includes therein an inflatable bladder that can be filled with air and thereby apply pressure to the forearm so as to cut off blood flow through the arteries to give a bloodless surgical field in the hand.

U.S. Pat. No. 5,133,734 issued to Ling H. Lee on Jul. 28, 1992, describes a pneumatically operated femoral artery compressor having a downward-pointing syringe plunger with a pressure disk at the distal end thereof, in which the thigh area at which appears the femoral artery is placed beneath that disk after transfemoral angiographic procedures, and pressure is applied to stop bleeding.

In summary, a number of the aforesaid patents, although involving pneumatic devices to be applied to the body, do not specifically address the issue of applying pressure to the dressings that overlie wounds, while the remainder are specifically related to certain surgical procedures or other circumstances in which the device described has been designed for the purpose and has no application elsewhere and particularly not to the circumstance that the invention addresses. What is needed and would be useful, therefore, is a general purpose apparatus that could be employed not only in certain ones of the circumstances described by those patents, but also in circumstances that none of them treat. A very important circumstance of that type lies in the occurrence of head wounds, which can bleed very profusely, but yet, to Applicant's knowledge, there exists no ready and convenient apparatus by which a truly functional pressure bandage could be applied to head wounds, or indeed in a variety of circumstances for which no specific device, of the types described in the foregoing patents, has been shown. For purposes of use by Emergency Medical Technicians (EMTs) in particular, to have such a device available would be of inestimable value, given that the EMTs have no way of knowing what kinds of wounds they might encounter on a shift, and in any event the ambulance or other EMT vehicle is limited in available storage space, even if there were available some array of pressure bandage systems that could be used in the wide variety of circumstances that might be encountered. Consequently, there is shown and described herein an apparatus, applicable to all circumstances that EMTs, “medics” out on the battlefield, or even lay persons in their own homes, vehicles or elsewhere might encounter, namely, a simple, general purpose pressure application device, or more specifically a pressure bandage, that can be used to apply pressure to a dressing that has been placed on any part of the body, and indeed for other purposes such as an emergency tourniquet or the like.

### BRIEF SUMMARY OF THE INVENTION

The present invention consists of a simple method and apparatus, designated as a pressure bandage, for applying pressure to the dressing on a wound at various parts of the body, consisting essentially of an air bladder, means for forcing air into the bladder and monitoring the pressure so achieved, means for releasing such air, and finally a bandage by which the air bladder can be held against a dressing that has been applied to a wound, or other similar circumstances, whereby upon installation of the air bladder against a dressing of a wound by use of the bandage, the forcing of air into the bladder will develop pressure against the dressing and hence against the wound, so as to staunch bleeding or for similar purposes.

### BRIEF DESCRIPTION OF THE SEVERAL VIEWS OF THE DRAWINGS

FIG. 1 shows an overall perspective view of a preferred embodiment of the invention.

FIGS. 2-5 show sequential cross-sectional views of a limb and a wound thereon onto which the embodiment of FIG. 1 is being applied.

### DETAILED DESCRIPTION OF THE INVENTION

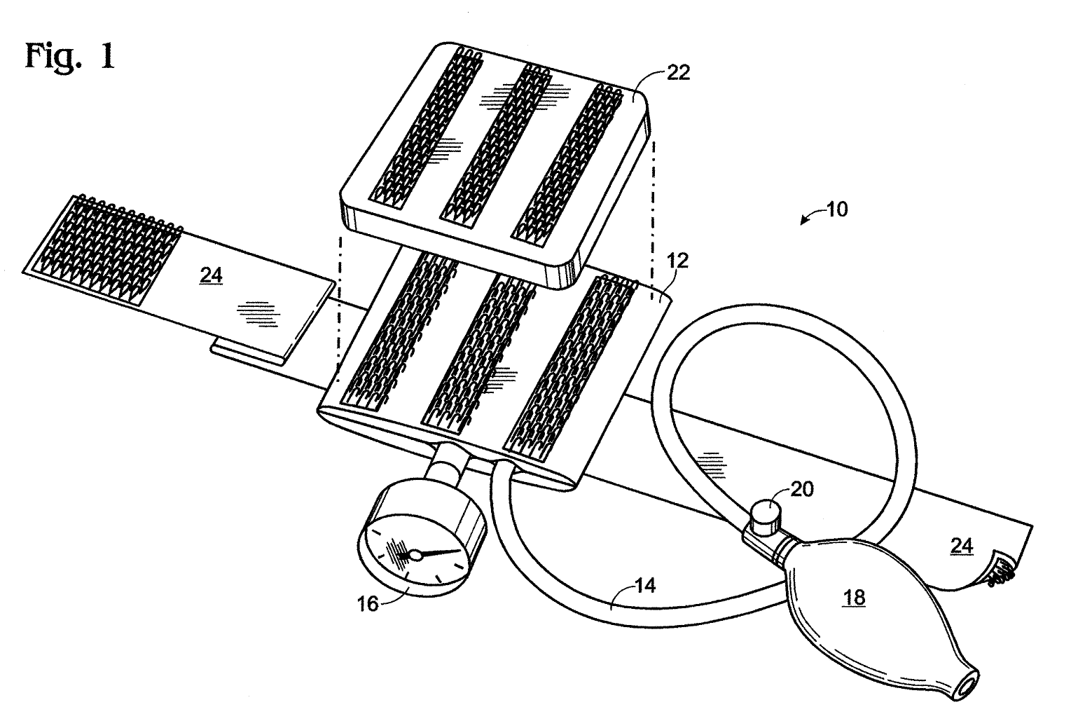

FIG. 1 shows in an overall perspective view a preferred embodiment of pressure bandage 10, wherein the several parts thereof have been laid out so as to suggest the manner of use. Pressure bandage 10 includes firstly an air bladder 12 to which is connected an air hose 14 so as to establish fluid connection therewith. Also connected to air bladder 12 in fluid connection is a pressure gauge 16, and at the distal end of air hose 14 there is connected, likewise in fluid connection, a bulb 18 by which air can be forced into air bladder 12. The pressure so created within air bladder 12 is measured by pressure gauge 16. Bulb 18 also has in fluid connection therewith a pressure release valve 20 by which pressure applied to a dressing (not shown) may be removed. Pressure bandage 10 further includes a pressure pad 22 and a bandage 24, wherein pressure pad 22 is to be placed over such a dressing on a wound or the like, and then bandage 24 is to be wrapped around the affected body part (not shown) so as to lie over that wound dressing or the like, and thereby to enable inflation of air bladder 12 so as to apply pressure to pressure pad 22 and hence to the dressing and wound.

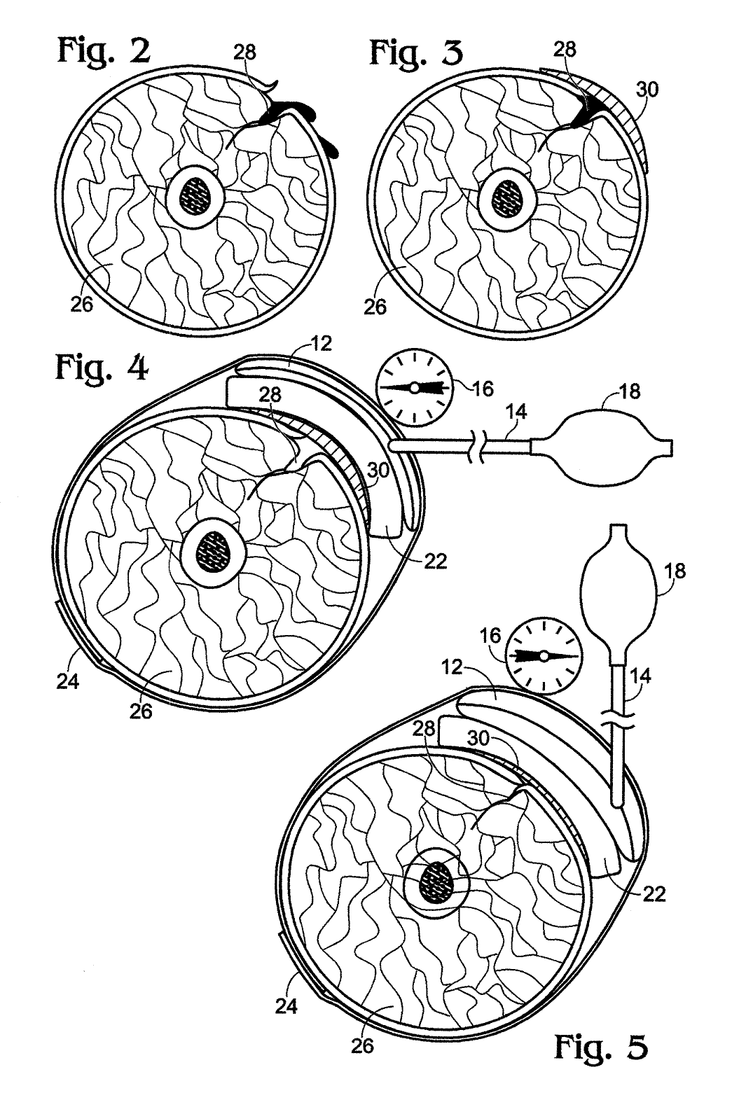

FIG. 2 shows in cross-section a body member 26 having a wound 28. The treatment of wound 28 commences with the application of a dressing 30 thereto as shown in FIG. 3. Then in FIG. 4 there is shown a complete installation of pressure bandage 10, wherein pressure pad 22 has been placed over dressing 30, which itself has been placed over wound 28 on body member 26, bladder 12 in an uninflated state has been placed over pressure pad 22, and bandage 24 has been wrapped around the entirety thereof and self connected at facing ends thereof. A low pressure, for purposes of illustration, has been shown on pressure gauge 18. Finally, FIG. 5 shows air bladder 12 having been inflated by bulb 18 through air hose 14, the higher air pressure so achieved in air bladder 12 being shown on gauge 16. In the configuration so shown in FIG. 5, pressure at the level shown on gauge 16 is being applied to pressure pad 22 and hence through dressing 30 to wound 28.

Pressure bandage 10 can be removed when blood flow has ceased, or at any other time that proper medical procedure so indicates. The air within air bladder 12 can be released by pressure release valve 20 (not shown in FIGS. 4-5 for simplicity in the drawings), and bandage 24 would be disconnected from itself and removed, to allow removal of air bladder 12 and pressure pad 22.

While pressure bandage 10 has been shown in use with respect to a wound 28 on a body limb 26 of FIGS. 4 and 5, it should be understood that such description and associated figures are for illustrative purposes only, pressure bandage 10 also being useful for other purposes. For example, in the case of an accident by which a lower leg has been damaged by an arterial wound, pressure bandage 10 might be used instead as an emergency tourniquet, to apply high and controllable pressure to the artery of the upper leg, where similar application to the wound itself would not be so effective in staunching blood flow.

In battlefield circumstances in particular, or in law enforcement work, it can happen that a person will have received a gun shot through an arm or leg, or even the torso, that leaves both entry and exit wounds. In that case, pressure bandage 10 can be adapted by the use of two air bladders 12 and pressure pads 22, to be placed over both wounds and then held in place by a single bandage 24. Since exit wounds are generally more damaging and critical than entry wounds, more pressure in the air bladder 12 that was applied to the exit wound would likely be required, and pressure bandage 10 is adaptable to having different pressures being used at different places, since it is largely air bladder 12 and not bandage 12 that determines the pressure in each case, even though some effect on a second pressure pad 22 when applying a greater pressure to a first air bladder 12 and pressure pad 22 would likely be seen. For this or similar circumstances in which more than one wound must be treated, pressure bandage 10 is sufficiently small, light and portable that several could be carried in a police vehicle, or indeed in the pack of a battlefield combatant.

1. A pressure bandage, comprising:
a pressure pad sized to be placed over a dressing on a bodily wound;
an air bladder adaptable to be placed over said pressure pad, distal from said dressing;
pressure application means adapted to generate air pressure within said air bladder;
pressure release means adapted to release air pressure from said air bladder; and
a bandage sized to be placed over said air bladder, pressure pad and dressing.

2. The pressure bandage of claim 1 wherein said pressure application means comprises an air hose connected at a proximal end to said air bladder, and a bulb attached to the distal end of said air hose.

3. The pressure bandage of claim 1 wherein said pressure release means comprises a release valve in fluid connection with said air hose and said bulb.
